(19) United States
(12) Patent Application Publication
(10) Pub. No.: US 2010/0286216 A1
(43) Pub. Date: Nov. 11, 2010

(54) COMPOSITION HAVING A HIGH OSMOTIC ACTIVITY AND EXHIBITING ANTIMICROBIAL, ANTI-INFLAMMATORY, AND REGENERATIVE EFFECTS

(75) Inventor: Boris Vitalyevich Stradomskiy, g. Rostov-na-Donu (RU)

Correspondence Address: COLLARD & ROE, P.C., 1077 NORTHERN BOULEVARD, ROSLYN, NY, 11576, US

(73) Assignees: Boris Vitalyevich STRADOMSKIY; Konstantin Mihailovich AVERIN; Yury Yuryevich SOLODUNOV

(21) Appl. No.: 12/735,245

(22) PCT Filed: Dec. 27, 2007
(86) PCT No.: PCT/RU2007/000745

§ 371 Date: Jun. 25, 2010

(51) Int. Cl. A61K 31/428; A61P 29/00; A61P 31/00; A61P 17/02

(52) U.S. Cl. 514/367

(57) ABSTRACT

The invention relates to pharmacology and cosmetology, in particular, to a group of medications containing iodine in active form, and may be used to treat septic wounds of different etiology, and also to prevent evolution of skin tissue pathology under the effect of varied injurious factors, including cosmetology.

The idea of the invention, which is a composition having a high osmotic activity and exhibiting antimicrobial, anti-inflammatory, and regenerative effects, said composition containing 1,3-diethylbenzimidazole triiodide as an active antimicrobial, anti-inflammatory, and regenerative principle, a solvent, medical low-molecular weight polyvinyl pyrrolidone as solubilizer and stabilizer of the active principle, is that the composition further contains polyethylene glycol (PEG or macrogol) having its own high pharmacological osmotic activity, and dimexide, and/or glycerin, and/or ethyl alcohol as the solvent, in the following proportions of the components, in percent by weight:

| | |
|---|---|
| 1,3-diethylbenzimidazole triiodide | 1.5-4.0 |
| Medical low-molecular weight polyvinyl pyrrolidone | 1.0-6.0 |
| Dimexide, and/or ethyl alcohol, and/or glycerin | 0.0-20.0 |
| Polyethylene glycol | The balance. |

### FIELD OF THE INVENTION

The invention relates to pharmacology and cosmetology, in particular, to a group of medications containing iodine in active form, and may be used for treating inflammatory process of various tissues and organs and in therapy of septic wounds, and in cosmetology.

### BACKGROUND OF THE INVENTION

The immediate prior art of this invention is the Betadin ointment containing Povidon iodine as an active principle and polyethylene glycol (macrogol) as the base (Instructions for Users of the Medication. Information for the Consumer—Betadin, an Ointment for External Use, Egis, Hungary).

The prior art medication, however, contains active iodine in the form of a simple complex only, in combination with polyvinyl pyrrolidone, for which reason it only has an antibacterial and anti-inflammatory effect, and no regenerative properties, and has a lower therapeutic activity as a result. Besides, the prior art medication may have an irritating effect caused by active iodine released un use in the wound from its complex with polyvinyl pyrrolidone.

In respect of the combination of essential characteristics of compositions used with the same purpose, the closet prior art is the invention of Russian Federation Patent No. 2,237,469 for “A Composition Displaying Anti-Inflammatory, Regenerative, and Antimicrobial Effects,” in which a composition displaying anti-inflammatory, regenerative, and antimicrobial effects contains 1,3-diethylbenzimidazole triiodide as an active principle, an organic dipolar dissociating (polar) basic soft aprotic solvent, medical low-molecular weight polyvinyl pyrrolidone, and a base, such as medical Vaseline or purified water (Patent RU No. 2,237,469, Nov. 18, 2003, IPC A 61 K 9/06, BI No. 28, Oct. 10, 2004).

The closet prior art invention is disadvantageous because it cannot be used against an evolving septic process. It uses medical Vaseline or purified water having a low osmotic activity as the base. In particular, the osmotic potential and osmolarity value of Vaseline are near 0. Osmolarity value shows the molar concentration of osmotically active particles in 1 kg of the solution. All medications used to treat a septic process, however, are to have a high osmotic activity to cause an intensive outflow of exudate from within the wound to the bandage, an antibacterial effect on infectious agents, rejection and melting of necrotic tissues, and evacuation of the wound content (L. A. Blatun, “Pharmacotherapy: Potentialities of Modern-Day Ointments in Treating Septic Wound, Bedsores, and Trophic Ulcers,” Pharmaceutical News, No. 3 (242), Jan. 29, 2002). Large quantities—at least 74.0% by weight—of Vaseline or water as the base of the closest prior art composition and no components displaying a high osmotic activity restrict significantly the use of the prior art composition in medical practice.

### SUMMARY OF THE INVENTION

The object of this invention that determines its purpose is to improve the efficiency of therapy of inflammatory processes in various tissues and organs, including treatment of septic wounds, and application in cosmetology.

The technical result that can be achieved by performing this invention consists in combining antimicrobial, anti-inflammatory, and regenerative effects of 1,3-diethylbenzimidazole triiodide as the active principle and a high osmotic activity of the composition as a result of pharmacologically active polyethylene glycol added to the composition.

The idea of the invention, which is a composition having a high osmotic activity and exhibiting antimicrobial, anti-inflammatory, and regenerative effects, and containing 1,3-diethylbenzimidazole triiodide as an active antimicrobial, anti-inflammatory, and regenerative principle, a solvent, and medical low-molecular weight polyvinyl pyrrolidone serving as solubilizer and stabilizer of the active principle, consists in that the composition comprises polyethylene glycol (PEG or macrogol) having its own high pharmacological osmotic activity and that dimexide, and/or ethyl alcohol, and/or glycerin are used as the solvent in the following proportions in percent by weight:

| | |
|---|---|
| 1,3-diethylbenzimidazole triiodide | 1.5-4.0 |
| Medical low-molecular weight polyvinyl pyrrolidone | 1.0-6.0 |
| Dimexide, and/or ethyl alcohol, and/or glycerin | 0.0-20.0 |
| Polyethylene glycol | The balance. |

The claimed invention is distinguished by that polyethylene glycol is added to the composition to, first, replace the emulsion system of Vaseline/active agent solution of the closest prior art invention with a truly therapeutically active solution of 1,3-diethylbenzimidazole triiodide that improves the pharmacological activity of the composition and, second, and most important, give the composition a high osmotic activity required to treat septic wound of different etiology.

The concentration range of composition components has been chosen for the following reasons. A concentration of 1,3-diethylbenzimidazole triiodide, the active antimicrobial, anti-inflammatory, and regenerative principle, below 1.5% by weight lowers the therapeutic activity of the composition. A concentration of 1,3-diethylbenzimidazole triiodide above 4.0% by weight does not increase the therapeutic effect, which results in unjustified waste of the active agent. Polyvinyl pyrrolidone concentration below 1.0% by weight does not achieve the required solubility of 1,3-diethylbenzimidazole triiodide in the hydrophilic medium of the wet septic wound. Polyvinyl pyrrolidone concentration above 6.0% by weight does not alter the solubility of 1,3-diethylbenzimidazole triiodide, and higher concentrations lead to unjustified waste of the ingredient. The concentration of solvents (dimexide, ethyl alcohol, and glycerin) and their proportions in the composition depend on the composition consistency (ointment, gel, solution, or suppositories) required in each specific case. When a dry composition is produced, no solvents are needed. Solvent concentrations above 20.0% by weight reduce the weight share of polyethylene glycol in the composition and, therefore, lead to undesired decline in osmotic activity of the composition. The choice of solvent type and combination of solvents depends on where the composition is used. In particular, it is reasonable to use dimexide or ethyl alcohol in medical practice. To prevent irritation of baby skin in pediatrics, glycerin is to be added. Glycerin is a suitable solvent in cosmetology.

Polyethylene glycol is used to give the composition a high osmotic activity needed and to obtain a true solution of 1,3-diethylbenzimidazole triiodide in quantities up to 100.0% by weight. The proportion of polyethylene glycol grades (200, 300, 400, 600, 1500, 3000, 4000, and 6000) depends on the required consistency of the composition to be produced.

A method for producing a composition of high osmotic activity exhibiting antimicrobial, anti-inflammatory, and regenerative effects may be carried out with the above purpose as follows. 1,3-diethylbenzimidazole triiodide (Specification 2492-003-40530251-05 or Pharmacopoeia Article 42-0609-6576-05) is used as an active antimicrobial, anti-inflammatory, and regenerative principle to produce a composition of desired quality and quantity. When liquid and soft composition forms are prepared with dimexide (Pharmacopoeia Article 42-2980-98), and/or ethyl alcohol (Pharmacopoeia Article FS 42-3072-00), and/or glycerin (Pharmacopoeia Article 42-2202-99 or GOST Standard 6824-96) that may be used as solvents, white medical low-molecular weight polyvinyl pyrrolidone powder (Pharmacopoeia Article 42-03-84-2668-02) used as solubilizer and stabilizer of the active principle is added to the mixture. The pharmacological activity of the resultant composition is not affected by the kind of solvent used. Dimexide and/or ethyl alcohol is used as solvent for preparing medications. Glycerin is used for making cosmetics. It is possible to use a mixture of solvents, for example, for preparing medications used in pediatrics to soften the effect of the composition. Finely ground dark-brown crystals of 1,3-diethylbenzimidazole triiodide are then added to the resultant mixture to obtain a therapeutically active composition displaying antimicrobial, anti-inflammatory, and regenerative effects, but lacking high osmotic activity. The resultant composition is mixed mechanically with liquid polyethylene glycol grades 200, 300, 400 or 600, or with a paste-like consistency polyethylene glycol that is a combination of grades 200, 300, 400 or 600 with grades 1500, 3000, 4000 or 6000, or with molten polyethylene glycol grades 1500, 3000, 4000 or 6000. The composition obtained as above displays a high osmotic activity as well as antimicrobial, anti-inflammatory, and regenerative effects.

For preparing dry composition forms (powders), finely ground crystals of 1,3-diethylbenzimidazole triiodide are mixed mechanically with white powder of medical low-molecular weight polyvinyl pyrrolidone. The resultant mixture that does not display high osmotic activity is mixed mechanically with finely ground polyvinyl pyrrolidone grades 1500, 3000, 4000 or 6000. The composition thus produced displays high osmotic activity as well as antimicrobial, anti-inflammatory, and regenerative effects.

The claimed method for producing compositions of high osmotic activity exhibiting antimicrobial, anti-inflammatory, and regenerative effects is safe for people involved in the production process. The examples that are given below to illustrate preparation of a composition having a high osmotic activity and displaying antimicrobial, anti-inflammatory, and regenerative effects are based on essential quality and quantity characteristics required and sufficient to produce the claimed composition having a high osmotic activity and displaying antimicrobial, anti-inflammatory, and regenerative effects, and have been used as a basis for specifications developed to produce the composition.

### EMBODIMENTS OF THE INVENTION

### Example 1

2.0% by weight of medical low-molecular weight polyvinyl pyrrolidone

(Mm 12,600 D) was dissolved in 5.0% by weight of dimexide as an organic solvent at 20° C. to 25° C. 3.0% by weight of 1,3-diethylbenzimidazole triiodide crystals were dissolved in the resultant solution. The resultant solution was added to 25.0% by weight of molten polyethylene glycol grade 1500 in 65.0% by weight of polyethylene glycol grade 400 at a temperature of or under 35° C. and stirred at cooling until the mixture reached a homogeneous state. The composition produced had a high osmotic activity (at osmolarity of approximately 1,800 mOsm/kg and osmotic potential in excess of 15 bar) and displayed antimicrobial, anti-inflammatory, and regenerative effects (ointment).

### Example 2

2.0% by weight of medical low-molecular weight polyvinyl pyrrolidone (Mm 12,600 D) was mixed with 9.0% by weight of glycerin as an organic solvent at 20° C. to 25° C. 3.0% by weight of finely ground crystals of diethylbenzimidazole triiodide were mixed with the resultant mixture. The resultant mixture was added to 25.0% by weight of molten polyethylene glycol grade 1500 in 61.0% by weight in polyethylene glycol grade 400 at a temperature of or under 35° C. and stirred at cooling until the mixture reached a homogeneous state. The composition produced as above had a high osmotic activity (osmolarity in excess of 2,600 mOsm/kg and osmotic potential over 15 bar) and displayed antimicrobial, anti-inflammatory, and regenerative effects (ointment).

### Example 3

1.0% by weight of medical low-molecular weight polyvinyl pyrrolidone (Mm 12,600 D) was dissolved in 4.0% by weight of dimexide as an organic solvent at 20° C. to 25° C., and 6.0% by weight of glycerin as an organic solvent was added to the mixture. 2.0% by weight of 1,3-diethylbenzimidazole triiodide crystals were dissolved in the resultant solution. The resultant solution was mixed with 87.0% by weight of polyethylene glycol grade 400 until a homogeneous state was reached. The composition produced had a high osmotic activity (osmolarity in excess of 2,800 mOsm/kg and osmotic potential over 15 bar) and displayed antimicrobial, anti-inflammatory, and regenerative effects (solution).

### Example 4

4.0% by weight of finely ground crystals of 1,3-diethylbenzimidazole triiodide were mixed mechanically with 6.0% by weight of medical low-molecular weight polyvinyl pyrrolidone (Mm 12,600 D) and 91.0% by weight of finely divided polyethylene glycol grade 1500. The composition produced had a high osmotic activity (osmolarity of approximately 600 mOsm/kg and osmotic potential over 15 bar) and displayed antimicrobial, anti-inflammatory, and regenerative effects (powder).

### Example 5

6.0% by weight of medical low-molecular weight polyvinyl pyrrolidone (Mm 12,600 D) was dissolved in 8.0% by weight of ethyl alcohol as an organic solvent at 30° C. to 35° C. 2.0% by weight of 1,3-diethylbenzimidazole triiodide crystals were dissolved in the resultant solution. The resultant solution was added to 25.0% by weight of molten polyethylene glycol grade 1500 at a temperature of or under 55° C. and stirred until a homogeneous state was reached. The mixture was poured into molds to produce suppositories and cooled to 0° C. The composition produced as above had a high osmotic activity (osmolarity in excess of 3,000 mOsm/kg and osmotic potential over 15 bar) and displayed antimicrobial, anti-inflammatory, and regenerative effects (suppositories).

All the compositions had pharmacological activity of antimicrobial, anti-inflammatory, and regenerative types in combination with high osmotic activity.

The materials and methods that can be used, as described above, to prepare compositions having a high osmotic activity and exhibiting antimicrobial, anti-inflammatory, and regenerative effects with the above purpose confirm conformity of the claimed invention to the Industrial Applicability patentability criterion.

What is claimed is:

1. A composition having a high osmotic activity and exhibiting antimicrobial, anti-inflammatory, and regenerative effects, said composition comprising 1,3-diethylbenzimidazole triiodide as an active antimicrobial, anti-inflammatory, and regenerative principle, a solvent, medical low-molecular weight polyvinyl pyrrolidone as solubilizer and stabilizer of the active principle, wherein the composition contains polyethylene glycol having a high osmotic activity, and dimexide, and/or ethyl alcohol, and/or glycerin as the solvent in the following proportions of the components, in percent by weight:

| | |
|---|---|
| 1,3-diethylbenzimidazole triiodide | 1.5-4.0 |
| Medical low-molecular weight polyvinyl pyrrolidone | 1.0-6.0 |
| Dimexide, and/or ethyl alcohol, and/or glycerin | 0.0-20.0 |
| Polyethylene glycol | The balance. |
